(19) United States
(12) Patent Application Publication
(10) Pub. No.: US 2017/0014569 A1
(43) Pub. Date: Jan. 19, 2017

(54) GEL DELIVERY CATHETERS, SYSTEMS, AND METHODS

(71) Applicant: BOSTON SCIENTIFIC SCIMED, INC., Maple Grove, MN (US)

(72) Inventors: AIDEN FLANAGAN, CO. GALWAY (IE); PHILIP BANNISTER, CO. LONGFORD (IE)

(73) Assignee: BOSTON SCIENTIFIC SCIMED, INC., MAPLE GROVE, MN (US)

(21) Appl. No.: 15/210,530

(22) Filed: Jul. 14, 2016

Related U.S. Application Data
(60) Provisional application No. 62/192,241, filed on Jul. 14, 2015.

(51) Int. Cl. A61M 5/155; A61M 5/32; A61M 25/00; A61M 5/19

(52) U.S. Cl. CPC A61M 5/155; A61M 5/19; A61M 5/3294; A61M 25/0074; A61M 2202/0007; A61M 2202/09; A61M 2210/125; A61M 2005/14513

(57) ABSTRACT

An injection catheter system is disclosed. The system includes a catheter defining a first pressure transfer lumen adapted to retain a pressure transfer material, an actuator at a proximal end of the catheter adapted to deliver a pressure from a proximal end to a distal end of the first pressure transfer lumen via the pressure transfer material, a distal section defining at least a first internal chamber adapted to retain a therapeutic gel component, at least a first plunger retained in the first internal chamber, and an injection port for delivering a therapeutic gel component into a treatment location from the first internal chamber when the actuator is used to deliver a pressure via the pressure transfer material to move the first plunger to deliver a therapeutic gel component through the injection port.

### CROSS-REFERENCE TO RELATED APPLICATIONS

This application claims priority under 35 U.S.C. §119(e) to U.S. Provisional Application No. 62/192,241, filed Jul. 14, 2015, the entire disclosure of which is herein incorporated by reference.

### TECHNICAL FIELD

This invention relates to delivering a therapeutic gel to a target area in a patient's body such as cardiac tissue.

### BACKGROUND

Heart failure due to damaged cardiac tissue is a significant health care issue. It has been proposed to treat the damaged tissue directly with a therapeutic agent designed to help regenerate the damaged tissue. An example of a therapeutic agent proposed for this use is stem cells. The stem cells would be delivered in the form of a gel to the site of the damaged tissue. The gels, however, can have relatively high viscosities. Therefore, administering the gel through a conventional syringe would subject the stem cells to relatively high pressure, potentially damaging the cells and compromising their therapeutic efficacy.

### SUMMARY

Methods, devices, and systems provided herein can deliver therapeutics, such as a gel including stem cells (e.g., cardiopoietic stem cells), to a treatment location (e.g., cardiac tissue). In some cases, methods, devices, and systems provided herein can deliver gels including stem cells without compromising their therapeutic efficacy. In some cases, methods, devices, and systems provided herein can deliver gels including stem cells with a limited amount of shear force exerted on the stem cells.

In Example 1, an injection catheter system includes a catheter defining at least a first pressure transfer lumen adapted to retain a pressure transfer material, an actuator at a proximal end of the catheter adapted to deliver a pressure from a proximal end of the first pressure transfer lumen to a distal end of the first pressure transfer lumen via the pressure transfer material, a distal section at the distal end of the catheter defining at least a first internal chamber adapted to retain a therapeutic gel component, at least a first plunger retained in the first internal chamber, a proximal end of the plunger being in fluid communication with the first pressure transfer lumen and adapted to move within the first internal chamber, and an injection port for delivering a therapeutic gel component into a treatment location from the first internal chamber when the actuator is used to deliver a pressure via the pressure transfer material to move the first plunger to deliver a therapeutic gel component through the injection port.

In Example 2, an injection catheter system of Example 1, wherein the pressure transfer material is a pressure transfer fluid.

In Example 3, an injection catheter system of Example 1, wherein the pressure transfer material includes at least one threaded wire extending from the proximal end to the distal end of the catheter and through a threaded aperture through the first plunger.

In Example 4, an injection catheter system of one of Examples 1-3, wherein the distal section further defines a second internal chamber adapted to retain a second therapeutic gel component.

In Example 5, an injection catheter system of Example 4, further including a second plunger retained in the second internal chamber, the second plunger being adapted to move in the second internal chamber.

In Example 6, an injection catheter system of Example 5, further including at least a second pressure transfer lumen in fluid communication with a proximal end of the second plunger, the actuator being adapted to deliver a pressure to the second plunger via a pressure transfer material when actuated.

In Example 7, an injection catheter system of Example 5, wherein the first pressure transfer lumen is in fluid communication with the proximal end of both the first and second plungers.

In Example 8, an injection catheter system of one of Examples 1-7, wherein the injection port is part of a distal cap that is detachable and reattachable to the distal section.

In Example 9, an injection catheter system of Example 8, further including an adaptor for connecting the detachable distal cap to the distal section of the catheter, the adaptor including a mixing chamber.

In Example 10, an injection catheter system of one of Examples 1-9, wherein the injection port, the adaptor, or a combination thereof includes a non-uniform cross-sectional shape or cross-sectional area in order to promote mixing.

In Example 11, an injection catheter system of one of Examples 1-10, wherein the first internal chamber and the first plunger have corresponding cross-sectional shapes, wherein the first internal chamber has a cross-sectional area that is no more than 5% greater than a cross-sectional area of the first plunger.

In Example 12, an injection catheter system of one of Examples 1-3, wherein the first internal chamber includes stem cells.

In Example 13, an injection catheter system of one of Examples 1-12, wherein the catheter has a diameter of at least 8 french.

In Example 14, an injection catheter system of one of Examples 1-13, wherein the injection port is an injection needle having a diameter of about 27 gauge.

In Example 15, an injection catheter system of one of Examples 1-14, wherein the system is a kit that includes a plurality of detachable distal caps each adapted to mix at least two therapeutic gel components.

In Example 16, an injection catheter system of one of Examples 1-15, further including a loading cap adapted to be secured to the distal section to deliver therapeutic gel components into internal chambers of the distal section.

In Example 17, a method of filling an injection catheter system between injections into a target area which includes taking the injection catheter system of Example 8 and removing the distal cap from the distal section, injecting at least a first therapeutic gel component including stem cells into the first internal chamber, and securing a new distal cap onto the distal section.

In Example 18, a method of filling an injection catheter system of Example 17, wherein the therapeutic gel component is injected by attaching a loading cap to the distal section.

In Example 19, an injection catheter system includes a catheter defining at least a first pressure transfer lumen adapted to retain a pressure transfer fluid, an actuator at a proximal end of the catheter adapted to deliver a pressure transfer fluid into a proximal end of the first pressure transfer lumen, a distal section at a distal end of the catheter defining at least a first internal chamber adapted to retain a therapeutic gel component, at least a first plunger retained in the first internal chamber, a proximal end of the plunger being in fluid communication with the first pressure transfer lumen and adapted to move within the first internal chamber, and an injection port for injecting a therapeutic gel component into a treatment location from the first internal chamber when the actuator is used to deliver a pressure transfer fluid into the first pressure transfer lumen to move the first plunger to deliver a therapeutic gel component through the injection port.

In Example 20, an injection catheter system of Example 19, wherein the distal section further defines a second internal chamber adapted to retain a second therapeutic gel component.

In Example 21, an injection catheter system of Example 20, further including a second plunger retained in the second internal chamber, the second plunger being adapted to move in the second internal chamber.

In Example 22, an injection catheter system of Example 21, further including at least a second pressure transfer lumen in fluid communication with a proximal end of the second plunger, the actuator being adapted to deliver a pressure transfer fluid into a proximal end of the second pressure transfer lumen when actuated.

In Example 23, an injection catheter system of Example 21, wherein the first pressure transfer lumen is in fluid communication with the proximal end of both the first and second plungers.

In Example 24, an injection catheter system of one of Examples 19-23, wherein the injection port is part of a distal cap that is detachable and reattachable to the distal section.

In Example 25, an injection catheter system of Example 24, further including an adaptor for connecting the detachable distal cap to the distal section of the catheter, the adaptor including a mixing chamber.

In Example 26, an injection catheter system of one of Examples 19-25, wherein the injection port includes a non-uniform cross-sectional shape or cross-sectional area in order to promote mixing.

In Example 27, an injection catheter system of one of Examples 19-26, wherein the first internal chamber and the first plunger have corresponding cross-sectional shapes, wherein the first internal chamber has a cross-sectional area that is no more than 5% greater than a cross-sectional area of the first plunger.

In Example 28, an injection catheter system of one of Examples 19-27, wherein the first internal chamber includes stem cells.

In Example 29, an injection catheter system of one of Examples 19-28, wherein the catheter has a diameter of at least 8 french.

In Example 30, an injection catheter system of one of Examples 19-29, wherein the injection port is an injection needle having a diameter of about 27 gauge.

In Example 31, an injection catheter system of one of Examples 19-30, wherein the system is a kit that includes a plurality of detachable distal caps each adapted to mix at least two therapeutic gel components.

In Example 32, an injection catheter system of Example 31, further including a loading cap adapted to be secured to the distal section to deliver therapeutic gel components into internal chambers of the distal section.

In Example 33, an injection catheter system which includes a catheter defining at least a first pressure transfer lumen adapted to retain at least one threaded rod, a distal section at a distal end of the catheter defining at least a first internal chamber adapted to retain a therapeutic gel component, at least a first plunger retained in the first internal chamber, a proximal end of the plunger being in fluid communication with the first pressure transfer lumen and adapted to move within the first internal chamber, the first plunger defining a threaded aperture there through, at least one threaded rod retained in the first pressure transfer lumen from a proximal end to the distal end and passing through the threaded aperture of the first plunger, an actuator at the proximal end of the catheter adapted to rotate the at least one threaded rod to advance the first plunger, and an injection port for injecting a therapeutic gel component into a treatment location from the first internal chamber when the actuator is used to rotate the threaded rod and advance the first plunger.

In Example 34, an injection catheter system of Example 33, wherein the distal section further defines a second internal chamber adapted to retain a second therapeutic gel component, a second plunger retained in the second internal chamber, the second plunger defining a second threaded aperture there through, and a second threaded rod extending from the proximal end to the distal end of the catheter and through the second threaded aperture.

In Example 35, an injection catheter system of one of Examples 33 or 34, wherein the injection port is part of a distal cap that is detachable and reattachable to the distal section.

In Example 36, an injection catheter system of one of Example 35, further including an adaptor for connecting the detachable distal cap to the distal section of the catheter, the adaptor including a mixing chamber.

In Example 37, an injection catheter system of one of Examples 33-36, wherein the injection port includes a non-uniform cross-sectional shape or cross-sectional area in order to promote mixing.

In Example 38, an injection catheter system of one of Examples 33-37, wherein the first internal chamber and the first plunger have corresponding cross-sectional shapes, wherein the first internal chamber has a cross-sectional area that is no more than 5% greater than a cross-sectional area of the first plunger.

In Example 39, an injection catheter system of one of Examples 33-38, wherein the first internal chamber includes stem cells.

In Example 40, an injection catheter system of one of Examples 33-39, wherein the catheter has a diameter of at least 8 french.

In Example 41, an injection catheter system of one of Examples 33-40, wherein the injection port is an injection needle having a diameter of about 27 gauge.

In Example 42, an injection catheter system of one of Examples 33-41, the system is a kit that includes a plurality of detachable distal caps each adapted to mix at least two therapeutic gel components.

In Example 43, an injection catheter system of Example 42, further including a loading cap adapted to be secured to the distal section to deliver therapeutic gel components into internal chambers of the distal section.

In Example 44, a method of filling an injection catheter system between injections into a target area includes taking the injection catheter system of Example 19 and removing the distal cap from the distal section, injecting at least a first therapeutic gel component including stem cells into the first internal chamber, and securing a new distal cap onto the distal section.

In Example 45, a method of filling an injection catheter system of Example 44, wherein the therapeutic gel component is injected by attaching a loading cap to the distal section.

In Example 46, a method of filling an injection catheter system between injections into a target area includes taking the injection catheter system of Example 33 and removing the distal cap from the distal section, injecting at least a first therapeutic gel component including stem cells into the first internal chamber, and securing a new distal cap onto the distal section.

In Example 47, a method of filling an injection catheter system of Example 46, wherein the therapeutic gel component is injected by attaching a loading cap to the distal section.

The injection catheter system delivers therapeutic gels having relatively high viscosities directly and accurately to a target site (e.g., damaged cardiac tissue) without subjecting the gels to forces that could damage the therapeutic agent and impair its efficacy. The gel can be pre-loaded in the injection catheter system or loaded by a physician at the time of use. The system can be used to deliver a single dose at a target site. Alternatively, the system can be used to deliver multiple doses at either a single site or multiple sites without re-loading.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates how an injection catheter system provided herein might be used to deliver gels including stem cells to a treatment location.

FIG. 2 illustrates an injection catheter system of FIG. 1 in greater detail with a magnified view of its distal end.

FIGS. 3A-3C are cross-sectional views of a various chamber configurations, which can be used in devices, systems, and methods provided herein.

FIGS. 4A and 4B are cross-sectional views of alternative distal tips of an injection catheter system having a distal tip with a spiral contour and a solid core.

FIG. 5 depicts an embodiment of an inner member including an inner port having a solid cylindrical core and helical ridge.

FIG. 6 illustrates a distal end according to certain embodiments of an injection device or system provided herein.

FIG. 7 illustrates an alternative embodiment of an injection device or system provided herein.

FIGS. 8A-8E illustrate an alternative embodiment of an injection device or system provided herein.

FIG. 9 illustrates an injection catheter system that includes a single chamber. Like reference symbols in the various drawings indicate like elements.

### DETAILED DESCRIPTION

Methods, devices, systems, and kits provided herein can deliver therapeutics, such as gels including tissues, biologics, stem cells, poietic cells, or fully diversified cells, to a treatment location, such a wall of a heart or other organ. The cells could be laboratory grown cells or cells from a donor. For example, cardiac cells could be injected into the heart tissue or liver cells injected into the liver.

In some cases, the therapeutics delivered using methods, devices, systems, and kits provided herein can have a relatively high viscosity, which can complicate the injection of these compositions. For example, therapeutics including cells when subjected to high pressures can experience high shear stresses, which can damage the cells, thus injecting such a composition through a long catheter lumen can require pressures that damage the cells. Methods, devices, systems, and kits provided here can use a pressure transfer material to transfer force to a therapeutic composition at a distal end of the injection device to inject the therapeutic composition, which can reduce the shear stress on the therapeutic composition. In some cases, the methods, systems, and devices provided herein can use the pressure transfer material to put less than 517 Pa stress on the therapeutic composition as the therapeutic composition is delivered. As used herein, “pressure transfer material” is a fluid or solid adapted to transfer pressure through a catheter, which may be bent in different directions, without significant pressure loss.

Methods, devices, and systems provided herein use a pressure transfer material to transfer pressure from a proximal end of a catheter to a plunger at a distal end of the catheter. The use of a plunger can reduce the stress placed on the therapeutic composition. In some cases, the plunger can put less than 517 Pa stress on the therapeutic composition. As used herein, a “plunger” is any solid body shaped to fit snugly within a chamber or lumen such that fluids do not bypass the solid body but allows for movement of the solid body within the chamber or lumen.

In some cases, the pressure transfer material can be a pressure transfer wire. The pressure transfer wire can be moved at a proximal end of the catheter to move the wire within the catheter lumen to move a plunger at a distal end of the catheter.

In some cases, the pressure transfer material is a pressure transfer fluid. In some cases, the pressure transfer fluid can have a lower viscosity than the therapeutic composition. By pressurizing or compressing the pressure transfer fluid (e.g., by activating a syringe), the pressure transfer fluid can move a plunger positioned in a cavity located at a distal end of an injection device or system provided herein to force a therapeutic composition out of the cavity and through a distal tip of the device or system provided herein. In some cases, the pressure transfer fluid has a viscosity of less than 100 cps and the therapeutic composition has a viscosity of greater than 1,000 cps. In some cases, the pressure transfer fluid has a viscosity of less than 50 cps and the therapeutic composition has a viscosity of greater than 5,000 cps. In some cases, the pressure transfer fluid has a viscosity of less than 10 cps and the therapeutic composition has a viscosity of 10,000 cps or more. In some cases, the pressure transfer fluid is saline, blood serum, or another physiologically relevant and/or compatible fluid. All viscosities discussed herein are viscosities at body temperature unless otherwise indicated. In some cases, viscosity can be determined using a standardized measurement protocol, such as ASTM D 2983.

In some cases, the therapeutics delivered using methods, device, systems, and kits provided herein can come as two or more components that are mixed at the site of injection. In some cases, the two or more components can cross-link at the site of the injection to form a gel. For example, some pre-gel therapeutics, which can be delivered using methods, systems, devices, and kits provided herein, can include two gel components that are intended to be mixed equally at the site of injection to crosslink to form a therapeutic gel. In some cases, one or more gel components can include cells (e.g., cardiopoietic stem cells). In some cases, one or both gel components can have a viscosity of 100 cps or greater. In some cases, one or both gel components can have a viscosity of 500 cps or greater, 1,000 cps or greater, 5,000 cps or greater, or 10,000 cps or greater. In some cases, both gel components can have the same viscosity. In some cases, the gel components can each have a viscosity less than the viscosity of a gel resulting from the mixture of the gel components. In some cases, a cross-linked gel resulting from two or more gel components being mixed can have a viscosity of greater than 10,000 cps, greater than 100,000 cps, or greater than 1,000,000 cps. In some cases, the cross-linked gel components resulting from two or more gel components can be a solid.

Mixing different gel components to create a higher viscosity gel or solid can result in the clogging of passages in an injection device or system. In some cases, devices and systems provided herein can include a detachable tip including intersecting channels that mix two or more gel components. In some cases, methods provided herein include a step of separating a detachable tip from the remainder of a device provided herein between injections into anatomical locations to clean it out and replace it. In some cases, methods provided herein include a step of separating a detachable tip from a remainder of a device provided herein between injections into anatomical locations to replace it with a new detachable tip. In some cases, systems provided herein can include multiple detachable tips each having intersecting channels to mix two or more gel components for each injection device. In some cases, kits provided herein can include at least 2 detachable tips for each injection catheter. In some cases, kits provided herein can include at least 3, at least 5, at least 8, or at least 10 detachable tips for each injection catheter.

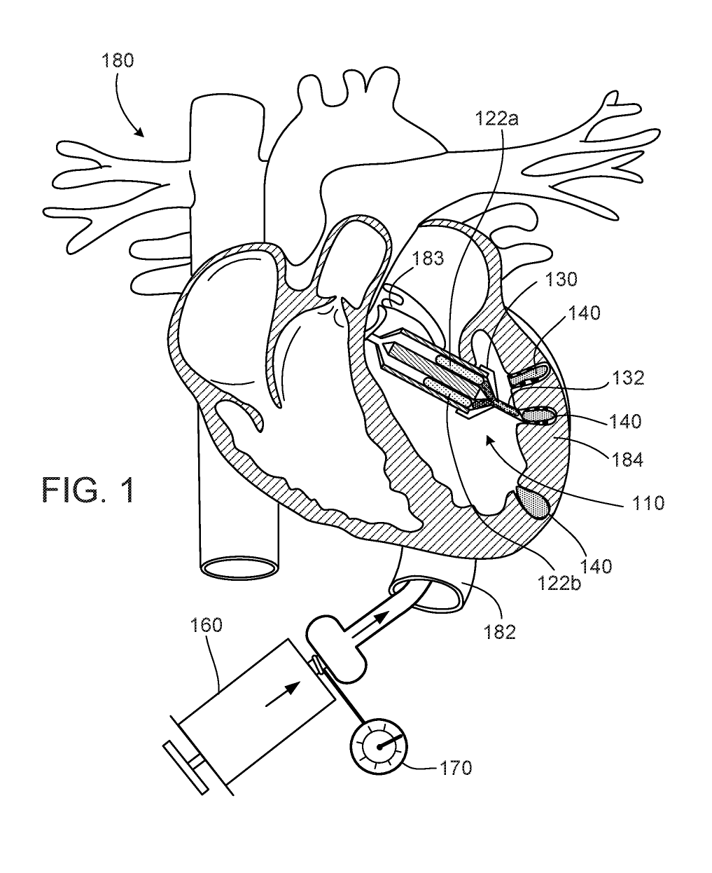

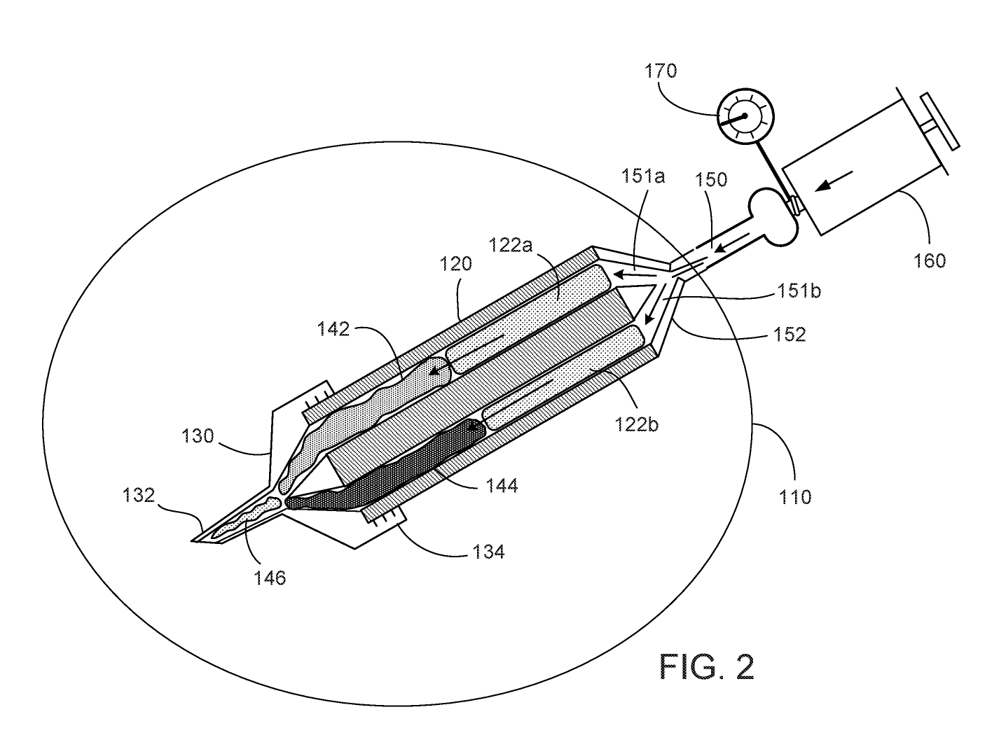

FIG. 1 illustrates how methods, devices, and systems provided herein can be used to deliver therapeutic agent deposits 140 to a left ventricle wall 184 of a heart 180 by advancing an injection catheter system 100 through the aorta 182 and the aortic semilunar valve 183. FIG. 2 depicts the catheter system shown in FIG. 1 in greater detail, with the distal end 110 being magnified. As shown, distal end 110 can include a distal section 120 and a distal tip 130.

Distal section 120 defines at least a first internal chamber. A first plunger 122a is retained in the first internal chamber. The first internal chamber can have a substantially uniform cross-sectional shape. The first internal chamber can have any suitable uniform cross-sectional shape, which can correspond to a cross-sectional shape of the first plunger 122a. In some cases, the first internal chamber and the first plunger 122a can each have a circular cross-sectional shape. In some cases, the first internal chamber and first plunger 122a can each have a semi-circular cross-sectional shape. In some cases, the area of the uniform cross-sectional shape of the first internal chamber is no more than 20% greater than the cross-sectional shape of first plunger 122a. In some cases, the area of the uniform cross-sectional shape of the first internal chamber is no more than 15% greater, no more than 10% greater, no more than 5% greater, or no more than 1% greater than the cross-sectional shape of first plunger 122a.

In some cases, distal section 120 can define at least a second internal chamber. A second plunger 122b is retained in a second internal chamber. In some cases, the second internal chamber can have the same cross-sectional area and/or cross-sectional shape as the first internal chamber. For example, in some cases, devices provided herein can include or be adapted to be filled with first and second therapeutic gel components intended to be mixed in equal parts. In some cases, the second internal chamber can have a different cross-sectional area and/or cross-sectional shape than the first internal chamber, which may be suitable for use with therapeutic compositions intended to be mixed in ratios other than 1:1. In some cases, additional internal chambers can also be included in distal section 120. In some cases, distal section 120 can include exactly 2 internal chambers. In some cases, distal section 120 can include 3 internal chambers, 4 internal chambers, 5 internal chambers, or 6 or more internal chambers.

The second internal chamber can have a substantially uniform cross-sectional shape. The second internal chamber can have any suitable uniform cross-sectional shape, which can correspond to a cross-sectional shape of second plunger 122b. In some cases, the second internal chamber and second plunger 122b can each have a circular cross-sectional shape. In some cases, the second internal chamber and second plunger 122b can each have a semi-circular cross-sectional shape. In some cases, the area of the uniform cross-sectional shape of the second internal chamber is no more than 20% greater than the cross-sectional shape of second plunger 122b. In some cases, the area of the uniform cross-sectional shape of the second internal chamber is no more than 15% greater, no more than 10% greater, no more than 5% greater, or no more than 1% greater than the cross-sectional shape of second plunger 122b.

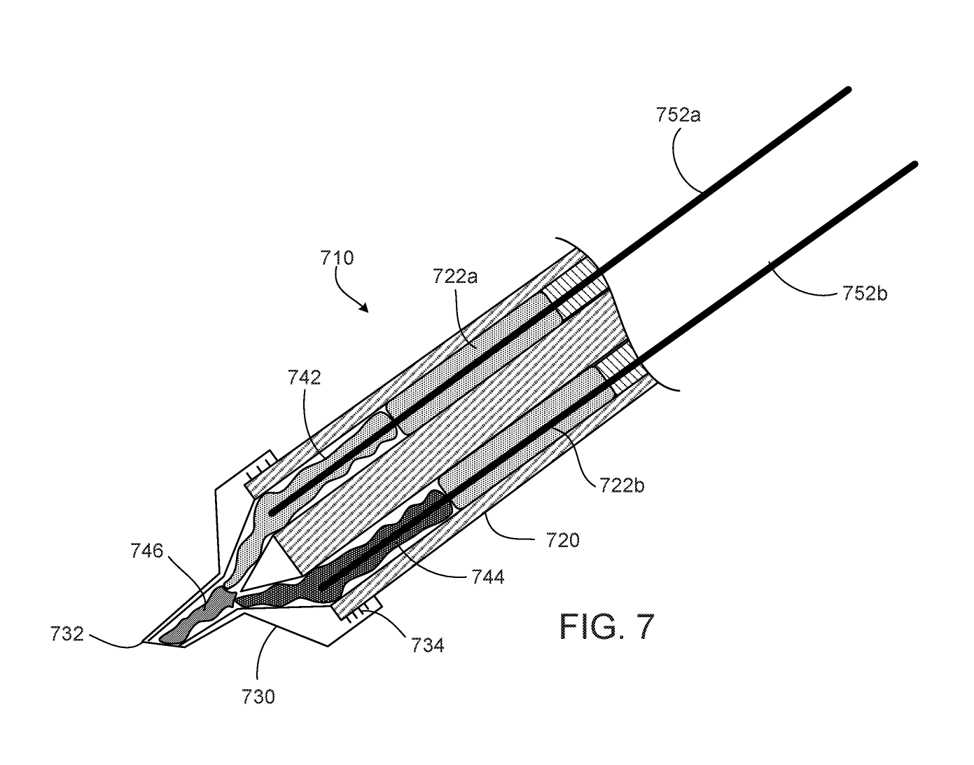

Injection catheter systems and devices provided herein include one or more pressure transfer lumens 151 extending from an actuator 160 (e.g., a fluid injector) at a proximal end to distal end 120 to transfer pressure from the proximal end of injection catheter system 100 to a proximal end of at least first plunger 122a (and in some cases second plunger 122b) to deliver one or more therapeutic gel components (e.g., components 142 and 144) in distal end 120. In some cases, the proximal end of systems and devices provided herein can include a pressure gauge 170. As shown in FIGS. 1 and 2, in some cases a single pressure transfer lumen 151 can include a fork 152 to deliver a pressure transfer fluid to a proximal side of two or more plungers (e.g., plungers 122a and 122b). In some cases not shown in FIGS. 1 and 2, injection catheter systems and devices provided herein can include separate pressure transfer lumens 151 each extending from adjacent injectors at a proximal end of a device or system provided herein to a different plunger in distal section 120. For example, FIG. 6 depicts a proximal end of a system having two fluid injectors 690a and 690b connected respectively to pressure transfer lumens 652a and 652b, which can each have a distal end abutting respectively a first plunger and a second plunger in a distal section (not shown).

Pressure transfer lumen in methods, devices, systems, and kits provided herein can contain or be adapted to contain any suitable pressure transfer fluid. In some cases, the pressure transfer fluid can be any physiologically relevant or compatible fluid. In some cases, the pressure transfer fluid can be saline. In some cases, the pressure transfer fluid can be water. In some cases, the pressure transfer fluid can be blood serum. In some cases, the pressure transfer fluid can have a viscosity of less than 100 cps, less than 50 cps, or less than 10 cps. In some cases, the pressure transfer fluid has a viscosity of about 1 cps. In some cases, the pressure transfer fluid can have a compressibility of less than 1×10⁻⁸ Pa⁻¹. In some cases, the fluid can be compressible. In some cases, the fluid can be a gas. In some cases, a compressible fluid can be used and the pressures in each lumen can be kept balanced. In some cases, a compressible fluid can be used and a fixed ratio of compressible fluid can be used to ensure a predetermined ratio of gel pre-components. As discussed above, the pressure transfer fluid can be used to transfer pressure to one or more plungers to deliver one or more therapeutic gels having a viscosity greater than the pressure transfer fluid, which can minimize the amount of pressure transferred to the therapeutic gel(s) and/or the shear stress on the therapeutic gel(s).

In use, for example, a distal tip 132 of catheter system 100 depicted in FIG. 1 can be positioned against left ventricular wall 184. In some cases, distal tip 132 can include radiopaque elements (not shown) used to ensure that it abuts heart wall 184. Once distal tip 132 is positioned adjacent to a treatment location, distal end 120 can be advanced to pierce into heart wall 184. After the needle advances, actuator 160 at a proximal end of injection catheter system 100 can be used to inject a pressure transfer fluid (e.g., saline) through pressure transfer lumen 151 to advance one or more plungers (e.g., 122a and 122b) to deliver a mixture of therapeutic gels 110 out through distal tip 132 to create a deposit 140 of therapeutic gel in left ventricular wall 184.

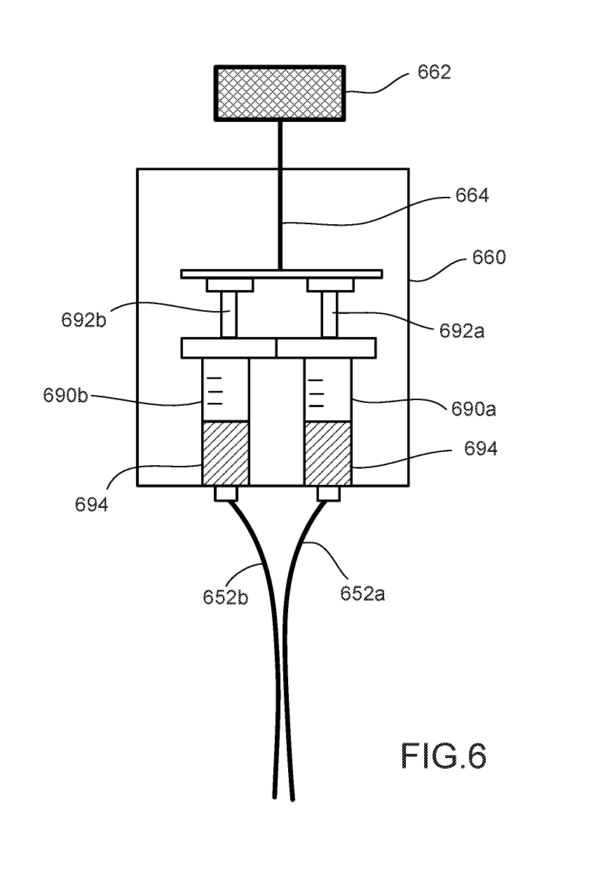

Clinicians can deliver therapeutics to treatment locations in a patient using methods, systems, devices, and kits provided. For example, a clinician can use a fluoroscopy or transesophageal ultrasonography that is connected to a video monitor to partially visualize a treatment location (e.g., the left ventricle). In some cases, an electrophysiology device (e.g., INTELLA, RHYTHMIA) can be used to monitor electrical activity on the ventricular wall and guide the delivery system to a site of low activity, which can identify damaged wall tissue for stem cell injection. In some cases, radiopaque marker bands can be implanted with the stem cells to ensure stem cells are implemented into the tissue wall of the heart. In some cases, when a clinician has positioned distal tip 132 against an inside surface of the left ventricular wall, the clinician can activate a catheter system 100 to deliver therapeutics 140. Between injections, catheter system 100 can be removed and refilled for a subsequent injection. An exemplary refilling process is discussed below in regards to FIG. 5.

In some cases, a distal cap 130 can include the distal tip 132. Distal cap 130 can include intersecting channels in fluid communication with internal chambers (e.g., 126 and 128) so that therapeutic gels (e.g., gels 142 and 144) can mix prior to injection into an anatomical location. In some cases, distal cap 130 can be removable from the remainder of distal section 120. In some cases, a clinician can remove the distal cap 130 between injections to clean it out and/or to replace it with a new cap to prevent clogging of the mixing channels. Any suitable locking mechanism can be used to connect the distal cap 130 to the distal section 120. In some cases, a spring loaded lock mechanism can be used to connect the distal cap 130 to the distal section 120. In some cases, distal cap 130 and/or distal section 120 can include teeth that are adapted to form a ratcheting mechanism with a squeeze release. In some cases, distal cap 130 can be integral with the portions of the distal section defining internal chambers (e.g., 126 and 128).

In use, a clinician can use actuator 160 (e.g., a fluid injector) to push a pressure transfer fluid through pressure transfer lumen 151. A pressure gauge 170 can detect a pressure within pressure transfer lumen 151 to detect a pressure applied to one or more plungers (e.g., 122a and 122b) in the distal section 120. In some cases, a proximal section of the catheter injector system 100 can include electronic or computerized controllers to regulate the injection force. In some cases, mechanical systems can be used to control the pressures provided by a fluid injector in actuator 160. Although there could be a small pressure drop because of losses in the system and small expansion of the catheter, catheter systems provided herein can be calibrated to correlate the actuator force to the gel injection force. In some cases, catheter systems provided herein can detect a force on the pressure gauge 170 and be adapted to halt the injection upon the discovery of a force in excess of a maximum, which may indicate a blockage, or below a minimum, which may indicate a leak.

Referring now to FIG. 2, catheter system 100 includes an actuator 160 (e.g., a fluid injector), a pressure gauge 170, a pressure transfer catheter 150 including one or more pressure transfer lumen 151, a distal end 120, and a distal cap 130. Pressure transfer catheter 150, distal end 120, and distal cap 130 can include any suitable polymeric or metallic material. For example, in some cases, pressure transfer catheter 150, distal end 120, and distal cap 130 can be made from polymeric materials such as, but not limited to, polytetrafluoroethylene (PTFE), fluorinated ethylene propylene (FEP), Hytrel®, nylon, Picoflex®, Pebax®, and the like. In some cases, pressure transfer catheter 150, distal end 120, and distal cap 130 can be made from metallic materials such as, but not limited to, nitinol, stainless steel, stainless steel alloys, titanium, titanium alloys, and the like.

Although FIGS. 1 and 2 depict a magnified view of distal end 120, in some cases the distal end 120 and the pressure transfer catheter 150 can have the same diameter. In some cases, distal end 120 and pressure transfer catheter 150 can have different diameters. In some cases, a pressure transfer lumen 151 leading to an internal chamber can have a different diameter or dimension such that a plunger (e.g., 122a or 122b) is restricted from entering pressure transfer lumen 151. In some cases, a transition from the pressure transfer catheter 150 to the distal end 120 can include a restriction in a lumen defining both a pressure transfer lumen and an internal chamber. Distal end 120 and pressure transfer catheter 150 can each have any suitable diameter. In some cases, distal end 120 and pressure transfer catheter 150 can have a diameter of 8 french. In some cases, distal end 120 and pressure transfer catheter 150 can have a diameter of 10 french.

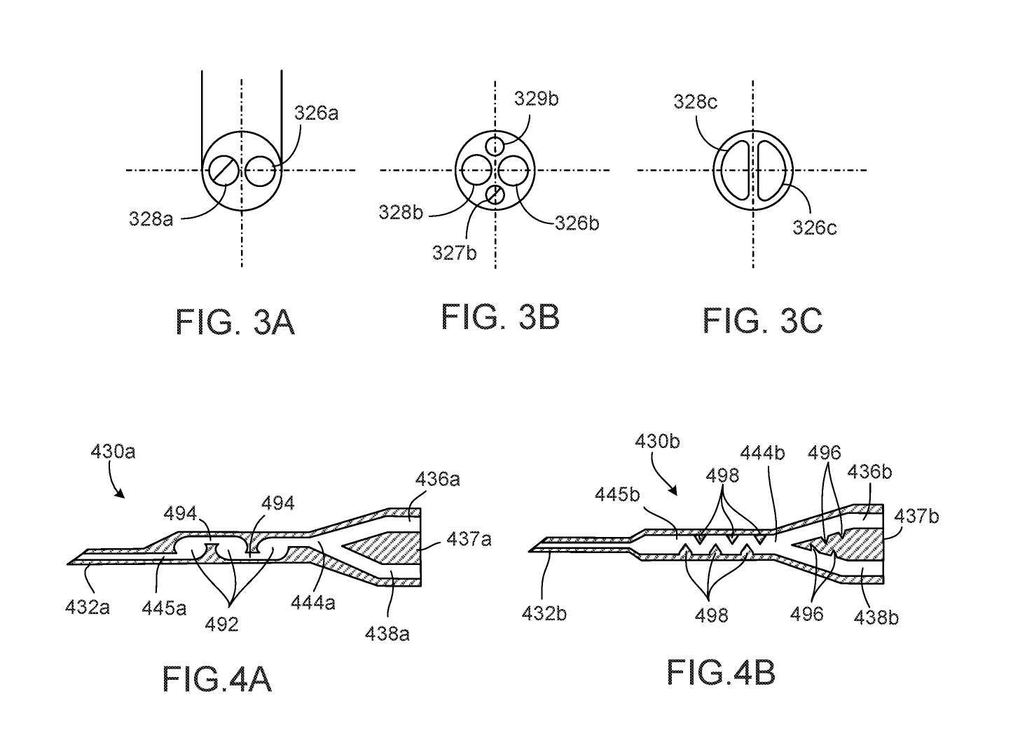

FIGS. 3A-3C depict cross-sections of distal end portions depicting different arrangements of internal chambers. These arrangements can also be used in the pressure transfer catheter 150 portion of the catheter injector system 100. FIG. 3A depicts a first internal chamber 326a and a second internal chamber 328a each having substantially circular cross-sectional shapes having the same dimensions. FIG. 3B depicts a first internal chamber 326b, a second internal chamber 327b, a third internal chamber 328b, and a fourth internal chamber 329b, with each having circular cross-sectional shapes but a first pair (326b and 328b) having a first diameter and a second pair (327b and 329b) having a second different diameter. FIG. 3C depicts a first internal chamber 326c and a second internal chamber 328c each having semi-circular cross-sectional shapes having the same dimensions.

Distal cap 130 as shown in FIGS. 1 and 2 includes an injection port 132, which can be an injection needle having a sharp distal edge to facilitate the piercing into a treatment location as shown. Injection port 132 can include a tubular metallic material. For example, in some cases, injection port 132 can be made from metallic materials such as, but not limited to, nitinol, stainless steel, stainless steel alloys, titanium, titanium alloys, and the like. Injection port 132 can be made in a variety of sizes to suit different applications. For example, in some cases a 27 gauge hypo tubing material is used to make injection port 132. In other cases, a 25 gauge, 22 gauge, or 19 gauge hypo tubing material is used to make injection port 132. Other larger or smaller sizes of tubing materials may also be used in some implementations. In some cases, the distal edge of injection port 132 can be beveled to create a sharp tip for penetrating tissue so that the port is a needle.

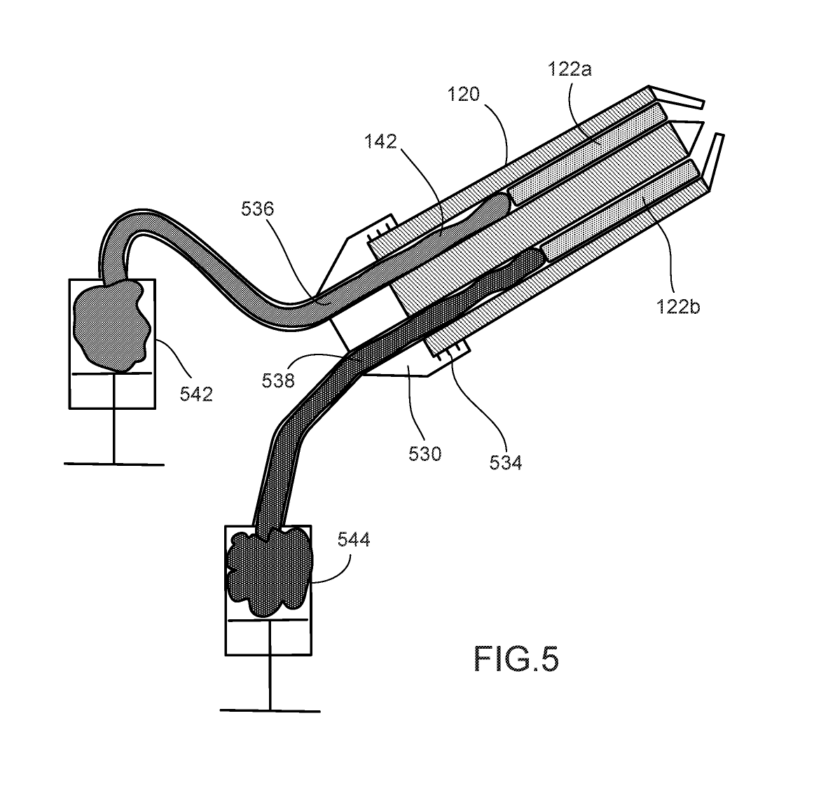

Distal cap 130 and injection port 132 can in some cases include channel features adapted to improve the mixing on two or more therapeutic gel components. FIGS. 4A and 4B depict distal caps 430a and 430b each including channel features adapted to improve the mixing of two of more therapeutic gel components pressed into the distal cap 130.

FIG. 4A depicts a distal cap 430a that includes channels 436a and 438a that intersect at 444a and then pass into mixing section 445a. As shown, mixing section 445a includes a series of larger diameter chambers 492 and smaller diameter channels 494 such that the change in diameter creates turbulence and mixing of the two therapeutic gel components. The mixture can then be injected though injection port 432a. High pressure in the small diameter to low pressure in the large diameter creates turbulence and therefore mixing due to an interruption of laminar flow, which keeps layers relatively static. In some cases, element 437a is left out.

FIG. 4B depicts a distal cap 430b that includes channels 436b and 438b that intersect at 444b and then pass into mixing section 445b. As shown, internal element 437b that partially defines channels 436b and 438b can include angled groves 496 adapted to cause a rotational movement of a therapeutic gel passing though channels 436b and 438b. In some cases, internal element 437b includes a helical groove 496. In some cases, a helical groove can also be included on the conical section of 430b that is opposite to that of helical groove 496. In some cases, fluid can rotate in one direction on the larger diameter surface and another direction close to 496 creating a mixing zone in between opposite helical grooves. In some cases, a helical groove can switch direction at one point causing extra turning and mixing of the fluid. Mixing section 445b includes a series of teeth 498 that create turbulence and mixing of the two therapeutic gel components. The mixture can then be injected though injection port 432b. High pressure in the small diameter to low pressure in the large diameter creates turbulence and therefore mixing due to an interruption of laminar flow, which keeps layers relatively static.

Injection catheter system 100 can be filled with therapeutic gel components 142 and 144 before each injection. FIG. 5 depicts an exemplary method of filling distal section 120 with therapeutic gel components before each injection. As shown, distal cap 130 is detached and a loading cap 530 is in its place. Loading cap 530 can be secured to distal section 120 via a locking mechanism 534 (e.g., a ratcheted threaded connection with a squeeze release). Loading cap 530 defines filling lumen 536 and 538 adapted to be aligned with first internal cavity 136 and second internal cavity 138 respectively and attached to syringes 542 and 544 respectively. After each injection, distal section 120 can be removed from a patient, distal cap 130 removed and loading cap 530 connected, and therapeutic gel components 142 and 144 reloaded. In some cases, therapeutic gel components 142 and 144 can include stem cells. Examples of useful gels include a first gel component including hyaluronic acid and hydrogen peroxide and a second gel component containing HA and horseradish peroxidase.

FIG. 6 depicts an actuator adapted to inject a desired ratio of pressure transfer fluids into different pressure transfer lumens. For example, as shown, a single actuator knob 662 can be rotated to actuate adjacent syringes 690a and 690b each including a pressure transfer fluid 694. As shown, a threaded bolt 664 can move a plate that presses against syringe plungers 692a and 692b in equal amounts to press equal amount of pressure transfer fluids 694 through pressure transfer lumen 652a and 652b. In some cases, if different amounts of pressure transfer fluids 694 in each line are desired, differently dimensioned syringes 690a and 690b can be used.

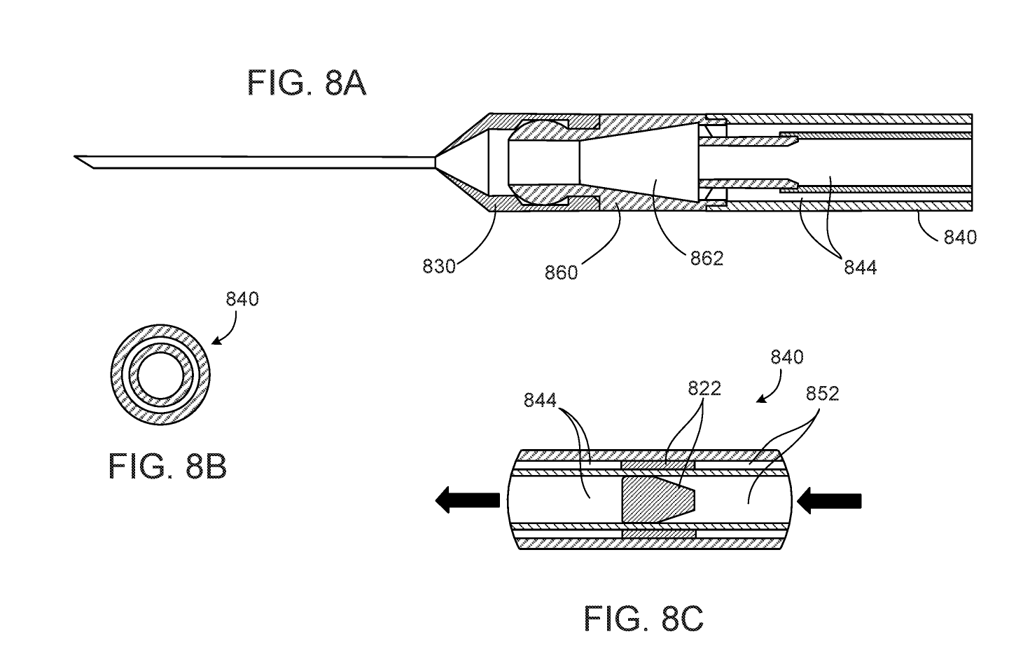

FIG. 7 depicts a distal end of an alternative catheter system for transferring pressure from a proximal end to a distal end of catheter 710 to deliver one or more gel components. As shown, distal section 720 defines at least a first internal chamber. A first plunger 722a is retained in the first internal chamber. The first internal chamber can have a substantially uniform cross-sectional shape. The first internal chamber can have any suitable uniform cross-sectional shape, which can correspond to a cross-sectional shape of the first plunger 722a. In some cases, the first internal chamber and the first plunger 722a can each have a circular cross-sectional shape. In some cases, the first internal chamber and first plunger 722a can each have a semi-circular cross-sectional shape. In some cases, the area of the uniform cross-sectional shape of the first internal chamber is no more than 20% greater than the cross-sectional shape of first plunger 722a. In some cases, the area of the uniform cross-sectional shape of the first internal chamber is no more than 15% greater, no more than 10% greater, no more than 5% greater, or no more than 1% greater than the cross-sectional shape of first plunger 722a.

In some cases, distal section 720 can define at least a second internal chamber. A second plunger 722b is retained in the second internal chamber. In some cases, the second internal chamber can have the same cross-sectional area and/or cross-sectional shape as the first internal chamber. For example, in some cases, devices provided herein can include or be adapted to be filled with first and second therapeutic gel components intended to be mixed in equal parts. In some cases, the second internal chamber can have a different cross-sectional area and/or cross-sectional shape than the first internal chamber, which may be suitable for use with therapeutic compositions intended to be mixed in ratios other than 1:1. In some cases, additional internal chambers can also be included in distal section 720. In some cases, distal section 720 can include exactly 2 internal chambers. In some cases, distal section 720 can include 3 internal chambers, 4 internal chambers, 5 internal chambers, or 6 or more internal chambers.

The second internal chamber can have a substantially uniform cross-sectional shape. The second internal chamber can have any suitable uniform cross-sectional shape, which can correspond to a cross-sectional shape of second plunger 722b. In some cases, the second internal chamber and second plunger 722b can each have a circular cross-sectional shape. In some cases, the second internal chamber and second plunger 722b can each have a semi-circular cross-sectional shape. In some cases, the area of the uniform cross-sectional shape of the second internal chamber is no more than 20% greater than the cross-sectional shape of second plunger 722b. In some cases, the area of the uniform cross-sectional shape of the second internal chamber is no more than 15% greater, no more than 10% greater, no more than 5% greater, or no more than 1% greater than the cross-sectional shape of second plunger 722b.

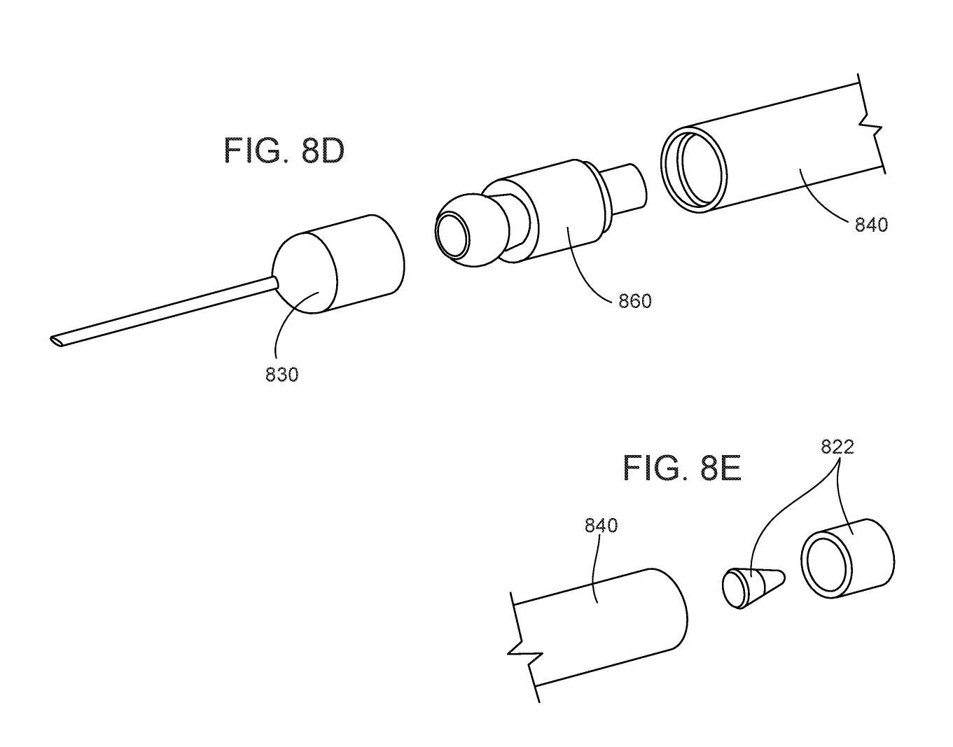

Injection catheter systems and devices provided herein include one or more pressure transfer lumens having one or more threaded wires 752a or 752b extending from an actuator (not shown) at a proximal end to distal end 720 to move plungers 722a and 722b. Plungers 722a and 722b each have a threaded aperture extending there through, and each threaded wire 752a and 752b extends through the threaded apertures such that the rotation of the wires causes the plungers to move in the first and second internal chambers. The actuator can cause the threaded wires to rotate to deliver one or more therapeutic gel components (e.g., components 742 and 744) in distal end 720. In some cases, the proximal end of systems and devices provided herein can include a pressure gauge (not shown). In some cases, the plunger actuation, by rotating threaded wires 752a and 752b, can be assisted by supplying a pressure via a pressure transfer fluid, as discussed above in regards to FIGS. 1 and 2. As shown in FIG. 7, components 742 and 744 can mix in removable cap 730 to form a therapeutic gel 746, which can be injected through port tip 732. The arrangement of the cap 730 can have the features discussed above in relation to FIGS. 3-6 and below in relation to FIGS. 8A-8E.

In some cases, rods 752a and 752b can instead be fixed to plungers 722a and 722b and when pressure is applied to an actuator (e.g., a syringe) outside the body the proximal position of the rods (at the actuator) indicates the amount of plunger movement and therefore volume of gel movement in each chamber.

FIGS. 8A-8E depict an alternative arrangement of a catheter system distal end provided herein. As shown, distal section 840 defines two concentrically arranged internal chambers 844 for retaining gel components. FIG. 8B shows a cross-sectional view of distal section 840 perpendicular to the length of distal section 840. In some cases, the concentric cross-sectional arrangement can extend from a distal end to a proximal end of the catheter. As shown in FIG. 8C, plungers 822 are retained in internal chambers 844. FIG. 8E depicts plungers 822 outside of the distal section 840, showing how one plunger is a circular ring and the other is plug shaped. FIGS. 8A and 8D depict how a distal cap 830 can be connected to distal section 840 via an adaptor 860. Adaptor 860 can include a mixing chamber 862. The mixing features discussed above in regards to FIGS. 4A and 4B can be included in mixing chamber 862 and/or in distal cap 830.

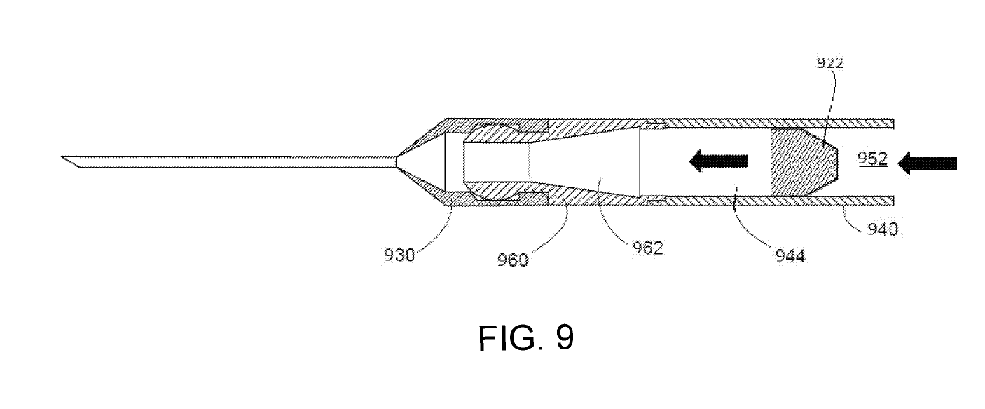

FIG. 9 illustrates an injection catheter system that includes a single chamber 944. In some cases, a therapeutic gel can be premixed prior to injection and be included in single chamber 944. As shown, distal section 940 defines a single internal chamber 944 for retaining a gel. A plunger 922 is retained in internal chamber 944. A distal cap 930 can be connected to distal section 940 via an adaptor 960. Adaptor 960 can include a mixing chamber 962. The mixing features discussed above in regards to FIGS. 4A and 4B can be included in mixing chamber 962 and/or in distal cap 930.

A number of embodiments of the invention have been described. Nevertheless, it will be understood that various modifications may be made without departing from the spirit and scope of the invention. For example, to aid in delivering the therapeutic gel to a specific treatment site of interest, the catheter injection system may be provided with a location device such as the IntellaTip MiFi™ XP available from Boston Scientific Corp. This device, which would be attached to the distal tip of the drug delivery catheter, features three mini-electrodes that provide accurate tip location and precise localized electrograms with minimal far-field effect. Accordingly, other embodiments are within the scope of the following claims.

What is claimed is:

1. An injection catheter system comprising:
(a) a catheter defining at least a first pressure transfer lumen adapted to retain a pressure transfer fluid;
(b) an actuator at a proximal end of the catheter adapted to deliver a pressure transfer fluid into a proximal end of the first pressure transfer lumen;
(c) a distal section at a distal end of the catheter defining at least a first internal chamber adapted to retain a therapeutic gel component;
(d) at least a first plunger retained in the first internal chamber, a proximal end of the plunger being in fluid communication with the first pressure transfer lumen and adapted to move within the first internal chamber; and
(e) an injection port for injecting a therapeutic gel component into a treatment location from the first internal chamber when the actuator is used to deliver a pressure transfer fluid into the first pressure transfer lumen to move the first plunger to deliver a therapeutic gel component through the injection port.

2. The injection catheter system of claim 1, wherein the distal section further defines a second internal chamber adapted to retain a second therapeutic gel component.

3. The injection catheter system of claim 2, further comprising a second plunger retained in the second internal chamber, the second plunger being adapted to move in the second internal chamber.

4. The injection catheter system of claim 3, further comprising at least a second pressure transfer lumen in fluid communication with a proximal end of the second plunger, the actuator being adapted to deliver a pressure transfer fluid into a proximal end of the second pressure transfer lumen when actuated.

5. The injection catheter system of claim 3, wherein the first pressure transfer lumen is in fluid communication with the proximal end of both the first and second plungers.

6. The injection catheter system of claim 1, wherein the injection port is part of a distal cap that is detachable and reattachable to the distal section.

7. The injection catheter system of claim 6, further comprising an adaptor for connecting the detachable distal cap to the distal section of the catheter, the adaptor comprising a mixing chamber.

8. The injection catheter system of claim 1, wherein the injection port comprises a non-uniform cross-sectional shape or cross-sectional area in order to promote mixing.

9. The injection catheter system of claim 1, wherein the first internal chamber and the first plunger have corresponding cross-sectional shapes, wherein the first internal chamber has a cross-sectional area that is no more than 5% greater than a cross-sectional area of the first plunger.

10. The injection catheter system of claim 1, wherein the first internal chamber comprises stem cells.

11. The injection catheter system of claim 1, wherein the catheter has a diameter of at least 8 french.

12. The injection catheter system of claim 1, wherein the injection port is an injection needle having a diameter of about 27 gauge.

13. The injection catheter system of claim 1, wherein the system is a kit that includes a plurality of detachable distal caps each adapted to mix at least two therapeutic gel components.

14. The injection catheter system of claim 12, further comprising a loading cap adapted to be secured to the distal section to deliver therapeutic gel components into internal chambers of the distal section.

15. An injection catheter system comprising:
(a) a catheter defining at least a first pressure transfer lumen adapted to retain at least one threaded rod;
(b) a distal section at a distal end of the catheter defining at least a first internal chamber adapted to retain a therapeutic gel component;
(c) at least a first plunger retained in the first internal chamber, a proximal end of the plunger being in fluid communication with the first pressure transfer lumen and adapted to move within the first internal chamber, the first plunger defining a threaded aperture there through;
(d) at least one threaded rod retained in the first pressure transfer lumen from a proximal end to the distal end and passing through the threaded aperture of the first plunger;
(e) an actuator at the proximal end of the catheter adapted to rotate the at least one threaded rod to advance the first plunger; and
(f) an injection port for injecting a therapeutic gel component into a treatment location from the first internal chamber when the actuator is used to rotate the threaded rod and advance the first plunger.

16. The injection catheter system of claim 15, wherein the distal section further defines a second internal chamber adapted to retain a second therapeutic gel component, a second plunger retained in the second internal chamber, the second plunger defining a second threaded aperture there through, and a second threaded rod extending from the proximal end to the distal end of the catheter and through the second threaded aperture.

17. The injection catheter system of claim 15, wherein the injection port is part of a distal cap that is detachable and reattachable to the distal section.

18. The injection catheter system of claim 15, wherein the injection port, the adaptor, or a combination thereof comprises a non-uniform cross-sectional shape or cross-sectional area in order to promote mixing.

19. An injection catheter system comprising:
(a) a catheter defining at least defining at least one internal lumen;
(b) a distal section at a distal end of the catheter defining at least a first internal chamber adapted to retain a therapeutic substance having a viscosity of greater than 10,000 cps;
(c) an injection port for injecting a therapeutic gel component into a treatment location from the first internal chamber; and
(d) a means for injecting the therapeutic substance through the injection port without subjecting the therapeutic substance to a stress greater than 517 Pa.

20. The system of claim 19, further comprising a therapeutic substance having a viscosity greater than 10,000 cps in the first internal chamber.
